(19) United States
(12) Patent Application Publication
(10) Pub. No.: US 2016/0256266 A1
(43) Pub. Date: Sep. 8, 2016

(54) INTRAOCULAR LENS WITH IMPROVED SHAPE RETENTION

(71) Applicants: Craig Alan CABLE, III, Mission Viejo, CA (US); Charles DeBoer, Sierra Madre, CA (US); Yu-Chong Tai, Pasadena, CA (US); Mark Humayun, Glendale, CA (US); Sean Caffey, Pasadena, CA (US)

(72) Inventors: Craig Alan CABLE, III, Mission Viejo, CA (US); Charles DeBoer, Sierra Madre, CA (US); Yu-Chong Tai, Pasadena, CA (US); Mark Humayun, Glendale, CA (US); Sean Caffey, Pasadena, CA (US)

(21) Appl. No.: 15/062,549

(22) Filed: Mar. 7, 2016

Related U.S. Application Data
(60) Provisional application No. 62/129,401, filed on Mar. 6, 2015.

(51) Int. Cl. A61F 2/16

(52) U.S. Cl. CPC A61F 2/1635; A61F 2002/1682; A61F 2002/169; A61F 2310/00011

(57) ABSTRACT

An accommodating intraocular lens has a central chamber with an optical axis extending through a vision-correcting optical zone and a peripheral region at least partially surrounding the optical zone but not interfering with light passing therethrough. At least one peripheral chamber surrounds the central chamber at least partially and functions as a storage reservoir for optical fluid, receiving excess fluid when the central chamber is compressed. Within each peripheral chamber (if there is more than one), a shape-retention member resists collapse of the peripheral chamber in response to external force.

### CROSS-REFERENCE TO RELATED APPLICATION

This application claims priority to, and the benefits of, U.S. Ser. No. 62/129,401, filed on Mar. 6, 2015, the entire disclosure of which is hereby incorporated by reference.

### FIELD OF THE INVENTION

In various embodiments, the present invention relates generally to implantable intraocular lenses and, more specifically, to intraocular lenses with structural features for improved use during implantation, accommodation, reaccess, and explantation.

### BACKGROUND

The crystalline lens of the human eye refracts and focuses light onto the retina. Normally the lens is clear, but it can become opaque (i.e., when developing a cataract) due to aging, trauma, inflammation, metabolic or nutritional disorders, or radiation. While some lens opacities are small and require no treatment, others may be large enough to block significant fractions of light and obstruct vision.

Conventionally, cataract treatments involve surgically removing the opaque lens matrix from the lens capsule using, for example, phacoemulsification and/or a femtosecond laser through a small incision in the periphery of the patient's cornea. An artificial intraocular lens (IOL) can then be implanted in the lens capsule bag—the sack-like structure remaining within the eye following extracapsular cataract extraction; the lens “capsule” is the thin clear membrane that surrounds the natural crystalline lens—to replace the natural lens. Generally, IOLs are made of a foldable material, such as silicone or uncrosslinked acrylics, to minimize the incision size and required stitches and, as a result, the patient's recovery time. The most commonly used IOLs are single-element lenses (or monofocal IOLs) that provide a single focal distance; the selected focal length typically affords fairly good distance vision. However, because the focal distance is not adjustable following implantation of the IOL, patients implanted with monofocal IOLs can no longer focus on objects at a close distance (e.g., less than 60 cm); this results in poor visual acuity at close distances. To negate this disadvantage, multifocal IOLs provide dual foci at both near and far distances. However, due to the optical design of such lenses, patients implanted with multifocal IOLs often suffer from a loss of vision sharpness (e.g., blurred vision, halos, glare, decreased contrast sensitivity). In addition, patients may experience visual disturbances, such as halos or glare, because of the simultaneous focus at two distances.

Recently, accommodating intraocular lenses (AIOLs) have been developed to provide adjustable focal distances (or “accommodations”), relying on the natural focusing ability of the eye (e.g., using contractions of ciliary muscles). Conventional AIOLs include, for example, a single optic that translates its position along the visual axis of the eye, dual optics that change the distance between two lenses, and curvature-changing lenses that change their curvatures to adjust the focus power. These designs, however, tend to be too complex to be practical to construct and/or have achieved limited success (e.g., providing a focusing power of only 1-2 diopters).

Consequently, much effort is devoted to developing IOLs that provide a high degree of accommodation and appropriate focusing power, and which can be easily manufactured and implanted in human eyes. There are many types of IOLs that are approved for use as well as in development. The most common IOLs are monofocal IOLs, multifocal IOLs, toric IOLs, and accommodating IOLs. Monofocal IOLs are not adjustable, and so provide vision at a specific focal plane. Multifocal IOLs, which provide simultaneous near and far focal points, were developed to solve this problem and provide the patient with an increased depth of visual field. Multifocal IOLs often provide near and far focusing percentages dependent on pupil size, i.e., the amount of near focus (add) varies with the pupil diameter. As a result, location relative to the pupil is critical for accurate functioning.

Toric IOLs correct astigmatism in the cornea and consequently require a specific angular orientation after implantation to ensure proper optical functioning. In the extreme case, when the toric is 90° from its intended angular position instead of correcting astigmatism, it increases astigmatism. Therefore, toric IOLs contain a fiducial marker to indicate the angular position of the IOL. After implantation, the toric IOL is rotated into the correct location. AIOLs focus in response to the eye's natural focusing muscle, the ciliary muscle. Like the youthful natural lens, these lenses eliminate the need for reading glasses. AIOLs often require coupling to the ciliary muscles of the eye or the lens capsular bag for actuation. Other AIOL designs monitor the pupillary diameter, or tonus of the ciliary muscles. Based on this they adjust, either passively by being acted upon by the muscles or actively as in the case of an electroactive lens.

In all scenarios the position of the IOL is critical for functioning. Tip, tilt, angular displacement or decentration reduce optical quality of the IOL. In addition, they can prevent a toric IOL, multifocal IOL, or accommodating IOL from functioning properly. In addition, maintaining the positioning of an IOL in the lens capsule is important after opening an aperture in the posterior capsule of the eye—for example, after the treatment for posterior capsular opacification. This additional opening in the lens capsule can lead to decentering of the lens, or possibly the lens falling out of the lens capsule into the posterior chamber of the eye.

To implant an intraocular lens, an incision is made in the cornea, followed by a capsulorhexis, where a portion of the lens capsule is removed to provide surgical access to the natural lens. Most often, a central 5-6 mm capsulorhexis is made. The lens is removed using phacoemulsification in a process of fragmenting and aspirating the lens from the lens capsule. Finally, an intraocular lens is implanted into the empty lens capsule. A fluid-filled AIOL can be inserted through a small incision (under 3 mm) since it can fully collapse on itself. Once the AIOL is inserted into the eye, the surgeon can manipulate it into the correct general orientation. The AIOL is then filled, following which it will self-center within the capsule bag. The surgeon can also help manipulate the AIOL into the center of the capsule bag, but the AIOL will tend to self-center due to the dome shape of the optical zone. The surgeon can then rotate the AIOL into correct alignment. This can be important in the case of toric and asymmetric lenses used to correct aberrations of a patient's eye. Asymmetric lenses have a specific required orientation.

Some AIOLs include one or more peripheral fluid chambers surrounding the optical region of the lens, and which do not contribute to vision correction. Instead, these chambers help anchor the AIOL within the capsular bag and provide a storage reservoir into which optical fluid can accumulate when the eye's natural focusing action compresses the AIOL; this arrangement may make the AIOL more responsive, since the eye's ciliary muscles are not opposed by an incompressible optical liquid (e.g., silicone oil) within a fixed volume. A drawback of this design, however, is the distortive effect these chambers can have on the optical region of the lens as the chambers fill with liquid. In effect, a structure designed to make the lens more permissive for focusing can actually undermine optical performance.

### SUMMARY

In various embodiments, the invention relates to an AIOL that corrects vision and is optically responsive to the natural focusing mechanism of the patient's eye. The AIOL may comprise a central chamber that effects vision correction when implanted, and contains an optical fluid (such as silicone oil). The central chamber has an optical axis (which will be aligned with the patient's visual axis) extending through a vision-correcting optical zone, and a peripheral region at least partially surrounding the optical zone but not interfering with light passing therethrough. At least one peripheral chamber surrounds the central chamber at least partially and functions as a storage reservoir for optical fluid, receiving excess fluid when the central chamber is compressed. Associated with (e.g., within or surrounding) each peripheral chamber (if there is more than one), a shape-retention member resists collapse of the peripheral chamber in response to external force.

Accordingly, in a first aspect, the invention relates to an intraocular lens comprising, in various embodiments, a membrane defining a central chamber for containing an optical fluid and, when filled, to provide vision correction when implanted in a patient's eye, where the central chamber has an optical axis extending through a vision-correcting optical zone of the central chamber and a peripheral region at least partially surrounding the optical zone; a membrane defining at least one peripheral chamber at least partially surrounding the central chamber along the peripheral region thereof and outside the optical zone, where the peripheral chamber is in fluid communication with the central chamber; and associated with the at least one peripheral chamber, a shape-retention member for resisting collapse of the associated peripheral chamber in response to external force. For example, the shape-retention member may maintain the peripheral chamber at either of two conformal states (e.g., two elliptical states with different long-axis lengths).

In various embodiments, the shape-retention member is at least one spring. The spring may extend from an anterior inner surface of the intraocular lens to a posterior inner surface thereof. In some embodiments, the spring is oriented parallel to the optical axis of the lens.

Alternatively, the shape-retention member may be one or more buckling members or a hinged arrangement of struts. In some embodiments, the struts comprise living hinges, and the lens may have an anchor integral with the peripheral-chamber-defining membrane to which at least one of the struts is affixed. The struts may be configured to form a three-dimensional scaffold with the peripheral-chamber-defining membrane stretched thereover.

In some embodiments, the shape-retention member retains the associated peripheral chamber in an ellipsoidal configuration. The shape-retention member may be fabricated from, for example, metal or a polymer.

In a second aspect, the invention pertains to an intraocular lens comprising, in various embodiments, a membrane defining a central chamber for containing an optical fluid and, when filled, to provide vision correction when implanted in a patient's eye, where the central chamber has an optical axis extending through a vision-correcting optical zone of the central chamber and a peripheral region at least partially surrounding the optical zone; and a membrane defining at least one peripheral chamber at least partially surrounding the central chamber along the peripheral region thereof and outside the optical zone, where the peripheral chamber is in fluid communication with the central chamber. The membrane defining the central chamber comprises a plurality of concentric regions having different expansion properties. For example, the membrane defining a central chamber may become more convex and increase in optical power in response to increasing pressure within the central chamber. Alternatively, it may become less convex and decrease in optical power in response to increasing pressure within the central chamber. In some embodiments, in response to increasing pressure within the central chamber, a first concentric region increases in convexity and a second concentric region increases in concavity. This may result, for example, from different thicknesses and/or material elasticities (e.g., Young's modulus). In other embodiments, in response to increasing pressure within the central chamber, a first concentric region decreases in convexity and a second concentric region decreases in concavity.

The term “substantially” or “approximately” means ±10% (e.g., by weight or by volume), and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 is a head-on elevation of a conventional fluid-filled intraocular lens.

FIGS. 2A and 2B show cross-sections of, respectively, a prior-art lens with conventionally circular chambers in the natural (non-accommodative) state within the capsule, and in a non-primary conformal (accommodative) state.

FIGS. 2C and 2D show cross-sections of, respectively, a lens with elliptical chambers in the natural (non-accommodative) state within the capsule, and in a non-primary conformal (accommodative) state in accordance with an embodiment of the invention.

FIGS. 3A and 3B are sectional views illustrating the effect of internal pressure on the lens.

FIGS. 4A, 4B, 5A, and 5B depict different embodiments of features that retain the lens or chamber thereof in an ellipsoidal shape.

FIGS. 6A and 6B depict embodiments of an arrangement with hinged struts.

FIG. 7A is a partial schematic of an embodiment in which an increase in fluid pressure alters the convexity of the optical zone to provide more optical power.

FIG. 7B is a partial schematic of an embodiment in which an increase in fluid pressure alters the convexity of the optical zone to provide less optical power.

FIGS. 8A and 8B are partial schematics of embodiments having membranes with regions of different thicknesses and/or elasticity.

### DETAILED DESCRIPTION

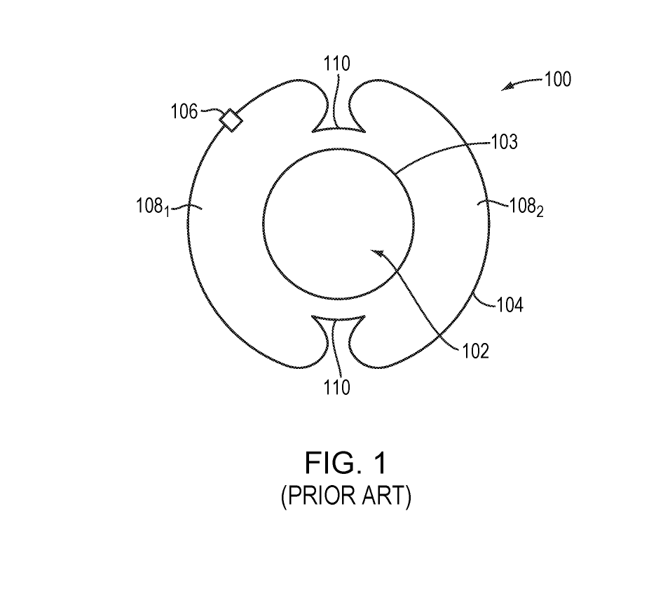

FIG. 1 depicts a liquid-filled accommodating IOL 100 having an interior region 102, which includes an optical zone 103 (with the optical axis passing through and perpendicular to the page) and is surrounded and defined by a membrane 104. The membrane 104 may be made of a flexible polymeric material such as silicone or parylene. A valve 106 facilitates filling and, in some embodiments, refilling of the AIOL 100 with an optical fluid.

As the interior region 102 fills with liquid, internal pressure forces the membrane 104 to assume a domed shape along the optical axis that focuses light to the back of the retina. As the pressure within the lens interior 102 increases, the optical zone 103 changes in diameter and its radius of curvature changes, thus changing the point at which light is focused and enabling the patient to see at different distances. Although in FIG. 1 the interior region 102 defines a generally spherical optical zone 102, the AIOL 100 can be non-spherical (e.g., an ellipsoid) to account for astigmatism and to help reduce optical aberrations (and any other optical properties that need adjustment).

The AIOL 100 is shaped to form a pair of opposed side chambers 108₁, 108₂ that provide haptic surfaces and store optical fluid; these may be defined, along with the interior region 102, by a single membrane, or may be one or more separate structures surrounding and in fluid communication with a central lens structure defining the interior region 102. Optical fluid is pumped between the optical zone 102 the and chambers 108 by fluctuating pressures within the AIOL 100—e.g., as a result of accommodative effort by the ciliary muscles of the eye that forces fluid from the chambers 108 and thereby deforms the optical zone 102 to shift the eye's focus. Although two chambers 108 are illustrated in FIG. 1, other embodiments may include a single chamber or more than two chambers. In some embodiments, the outer edges 110 of the optical zone 102 are reinforced to discourage the side chambers 108 from straining, and thereby altering the optical character, of the optical zone. Unfortunately, such reinforcement may be insufficient and/or interfere with the lens's accommodative properties.

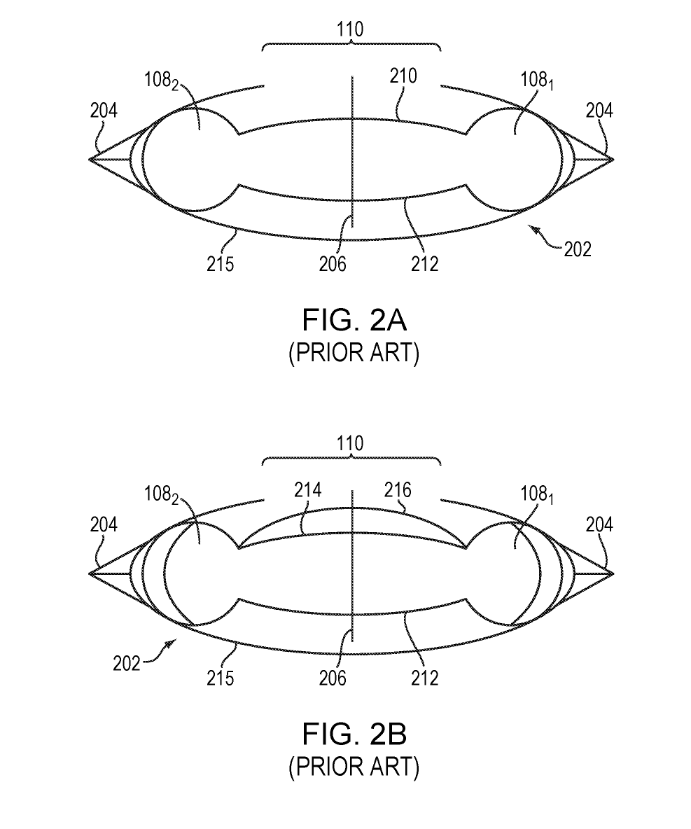

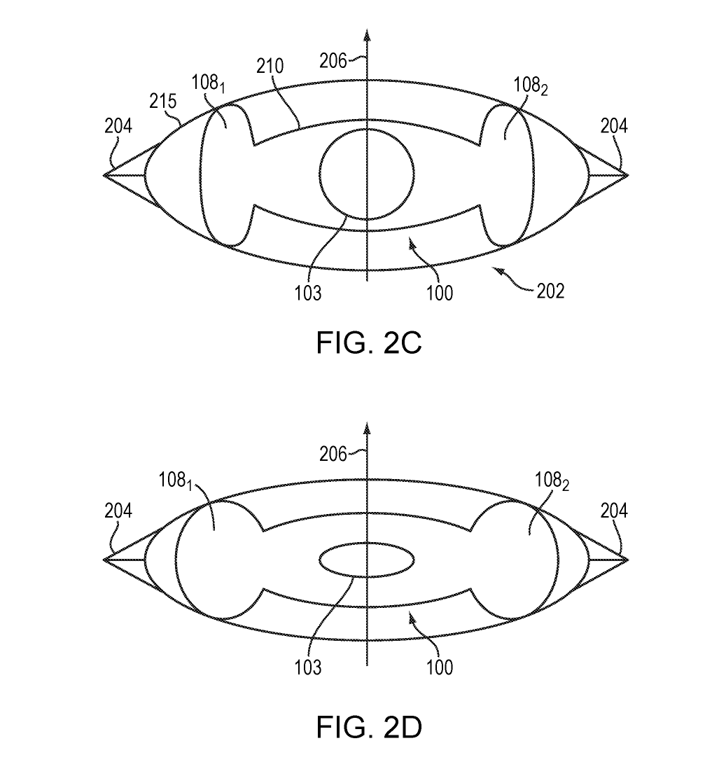

FIG. 2A shows an AIOL with conventionally circular chambers 108 in the natural (non-accommodative) state within the capsule 202 of a patient whose cataract has been removed, and with the optical axis 206 oriented as indicated. The anterior and posterior membranes 210, 212 of the optical zone 110 of the AIOL have a curvature that determines how light is focused for seeing at different distances, and may or may not be in contact with the capsule bag 215. If so, the curvature of the optical membranes 210, 212 allows the lens to center itself inside of the capsule bag 215. But it is the chambers 108 that provide the primary contact surfaces between the AIOL 100 and the interior of the capsule bag 202. More specifically, the primary contact surface between the AIOL 100 and the interior of the capsule bag 202 relies on equatorial alignment (i.e. contact with the capsular bag directly near the zonules 204) and radial compression.

The zonules 204 lie at the edges of the capsule bag 202, which interfaces (i.e., makes contact with) the lateral portions of the circular chambers 108. FIG. 2B shows the AIOL with the circular chambers 108 compressed as the zonules 204 relax and lateral force is transmitted through the capsular bag 215. This type of AIOL usually has at least one rigid membrane (in this case, the posterior membrane 212) to minimize lateral compression of the optical zone 110 and maximize lateral compression of the circular chambers 108 to force fluid into the optical zone 110. The non-rigid membrane portion 214 is thereby deflected into conformal state 216 to change the optical parameters of the lens. Once the zonules 204 tighten, the non-rigid membrane 214 returns to its natural conformation as shown in FIG. 2A.

FIGS. 2C and 2D show the contrasting behavior of an AIOL with elliptical side chambers 108 embodying an implementation of the present invention. This AIOL relies on compression of the long axis (depicted as the vertical axis for convenience) by flattening of the capsular bag 202 as the zonules 204 tighten; it does not rely on equatorial alignment (i.e., contact with the capsular bag directly near the zonules 204) and radial compression as is the case for the conventional design shown in FIGS. 2A and 2B. The compression force must be translated correctly, although the capsulexis procedure creates an opening on the anterior portion of the capsular bag 202 that is smaller than the fully expanded AIOL and varies from patient to patient. The AIOL is therefore implanted in an evacuated configuration (e.g., the interior region is minimally filled prior to implantation and assumes a saddle-like shape) that fits through the capsulexis opening. Post-implantation, a valve on one or both of the side chambers 108 (or the interior chamber outside the optical zone 110) is accessed to fill the AIOL. According to the capsule size, the AIOL may be filled with 30 to 280 μL, and more typically 80 to 170 μL, of optical fluid to maximize the region of contact between the side chamber 108 and lens capsule 202.

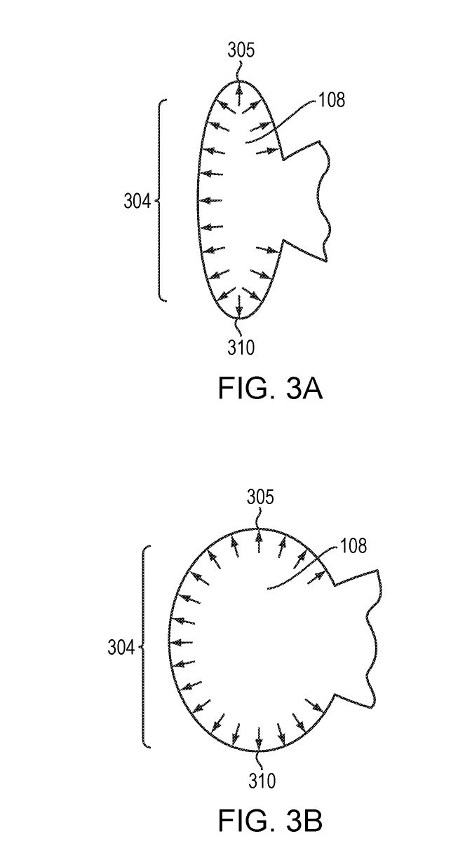

The effects on the chambers 108 of the pressures changes that result from the accommodative activity of the eye are illustrated in FIGS. 3A and 3B, with arrows pointing against the chamber membrane 302 representing the forces caused by the internal pressure of the fluid inside. FIG. 3A illustrates the configuration where relaxation of the zonules (causes release of fluid into the optical zone, while FIG. 3B shows the result of zonule tension that forces fluid into the chambers 108. As a qualitative indication of the magnitudes of the internal forces, FIG. 3A shows that there is greater force—represented by more arrows—against the side 304 of the chamber 108 than against the anterior and posterior regions 305, 310 of the membrane. This internal pressure profile urges the chamber 108 toward the more spherical conformation illustrated in FIG. 3B. If this is allowed to occur, however, the result will be unwanted distortion of the optical zone—i.e., distortion unrelated to that properly obtained as a result of the eye's natural focusing action, notwithstanding reinforcement of the regions 110 (see FIG. 1) of the AIOL 100. According to the magnitude of the pressure profile and the response of the capsular bag, there may be a permanent myopic or hyperopic shift. To some degree, such a shift may be corrected by accessing and altering the volume of filling fluid though one or more externally accessible valves as discussed above and/or by selecting or altering (e.g., via titration with a doping agent) the refractive properties of the optical filling fluid. Alternatively, the chambers 108 can be reinforced to favorably retain an ellipsoidal shape under pressure as further detailed below.

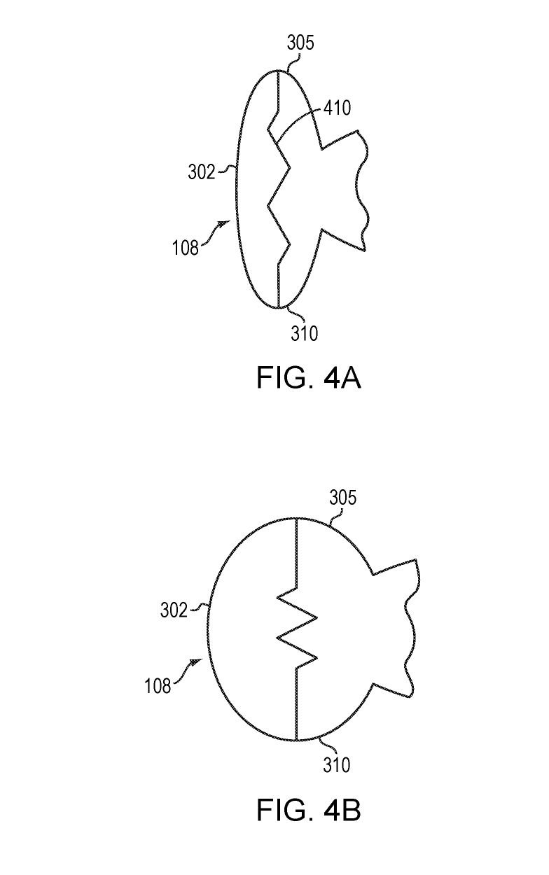

FIGS. 4A-6C illustrate different mechanical strategies for retaining this ellipsoidal conformation. As noted, preventing the conformation from becoming excessively circular avoids dependence on equatorial alignment which, when not achieved, causes interference with the optical properties of the lens. Another benefit is mitigating the anatomical effects that cataract surgery has on the lens capsule, namely, a tendency to change in size. Because of its asymmetry, an ellipsoid conformation also assist in initial implantation of the lens. It is generally difficult to match the lens to the diameter of the lens capsule because the latter is occluded by the iris from direct visual measurement; although ultrasound or MRI may be used to characterize the lens capsule, the obtainable imaging resolutions may not be sufficiently accurate. An ellipsoidal confirmation can be more responsive to the zonules, since its long axis is aligned with the axis of lens capsule flattening as it accommodates.

In FIGS. 4A and 4B, a spring 410 runs between the anterior and posterior regions 305, 310 and urges them apart, thereby augmenting the natural resistance of the membrane 302 to deformation. In some embodiments, the spring 410 is parallel to the optical axis of the lens. The spring 410 resists compression as shown in FIG. 4B, allowing for some deformation of the chamber 108 but, the more circular the chamber becomes (with concomitantly adverse effect on the optical configuration of the AIOL 100), the more resistance the spring 410 offers in accordance with Hooke's Law. Alternatively, the spring 410 may be configured to offer a non-linear resistance consistent with the anatomical behavior of the zonules during accommodation. The spring 410 may be made of metal (e.g., titanium or aluminum), polymer (e.g., an acrylate, crosslinked polyethylene, PMMA), or other suitable material, and may be introduced into the side chambers 108 during manufacturing by, for example, insert molding into the chamber portions, having the material (e.g., silicone) of the side chambers spin coated, or other suitable method. None of the mechanical features described herein prevents fluid continuity within the side chamber or between the side chamber and the interior region of the AIOL.

In another embodiment, the spring member 410 at least partially surrounds the chamber 302 externally, e.g., as a vertically oriented band or collar following at least a portion of the vertical circumference of the chamber. The vertical band or collar has a stiffness that resists deformation in the manner of a spring, and has a rest conformation corresponding to the elliptical shape of the chamber 302. The band or collar may, if desired, be overmolded by an additional coating (e.g. silicone, parylene).

In still other embodiments, the spring 410 is a buckling member—i.e., a column, piston or corrugated diaphragm that buckles recoverably under compressive axial load. In this case the force exerted by the member 410 does not vary linearly with compression. Instead, the member 410 prevents any shape distortion of the chamber 108 until the critical buckling load is reached. This load is set to discourage but not prevent a change in chamber shape. Although the figure shows simplified struts for ease of illustration, the contact points between the membrane and hinges may have continuity and suitable smoothing structures to minimize any puncturing forces. Specific spring designs such as a C-spring, S-spring or wave-spring may be employed, for example.

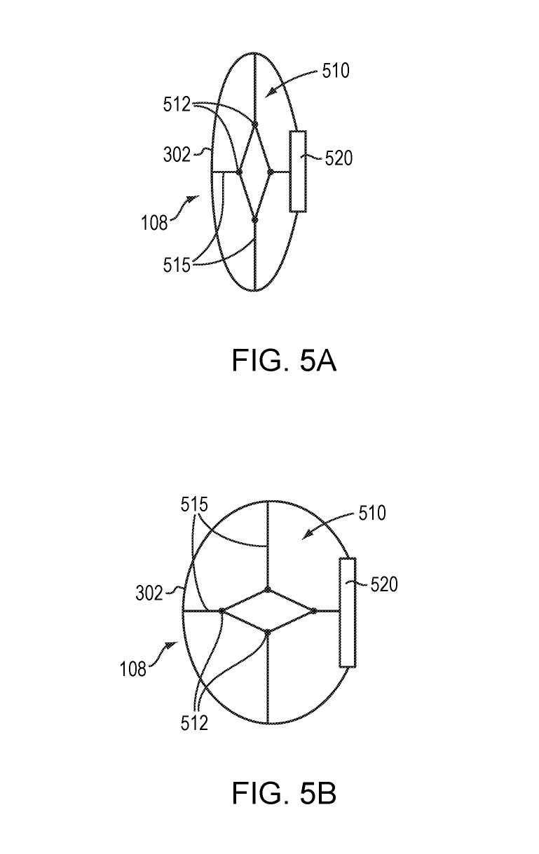

As shown in FIGS. 5A and 5B, shape retention can also be maintained by a hinged strut arrangement 510. The illustrated arrangement 510 includes four rigid struts arranged in a diamond configuration and joined by hinges 512, which may be, for example, living hinges (i.e., flexible hinges made from the same material as the struts—e.g., formed by thinning the material). The hinges exhibit sufficient stiffness that the strut arrangement resists deformation into the configuration shown in FIG. 5B, but once again, to prevent damage or patient injury, the structure 510 does not resist collapse to the point of risking puncture to the membrane 302. Alternatively, the hinges may rotate during compression in a multi-axis fashion but may be bridged by springs that resist compression. In another alternative, the arrangement may be a single, unitary loop of material (e.g., a rigid polymer band) that behaves as a spring, and may span the entire vertical extent of the chamber 108. Once again, the struts may be made of metal such as steel or aluminum, or a stiff polymer such as an acrylate or crosslinked polyethylene, or a shape-memory alloy (e.g., an engineering material such as a NiTi alloy) or polymer (e.g., polyurethane).

The strut arrangement 510 is retained within the chamber 108 by a series of support members 515 that reach, and in some embodiments are mechanically or adhesively affixed to, the interior surface of the chamber 108. These support members 515 may be made of the same material as that of the struts or a different material. To prevent movement of the strut arrangement 510 and support members 515 within the chamber 108, one or more of the support members 515 may be affixed to an anchor 520 integral with (i.e., affixed to or embedded within) the membrane 302. The anchor 520 may also participate in the mechanical action of the strut arrangement 510. In some embodiments, the anchor is a stiff ring through which optical fluid can pass. The anchor may additionally incorporate features of a flow restrictor or check valve to control the flow of fluid between the lens interior region 102 and one or more chambers 108 (see FIG. 1).

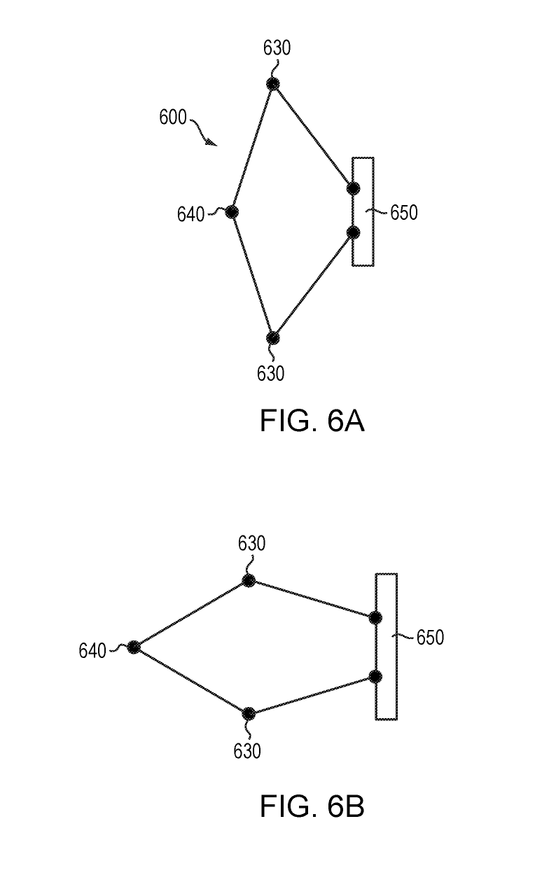

FIG. 6A and 6B depicts an embodiment in which the hinged strut arrangement 600 forms a scaffold that defines the shape of the chamber 108, so while this shape has a long and a short axis, it is not ellipsoidal. For example, instead of having a separate chamber membrane 302, the chamber membrane may be part of the scaffold 600 itself, extending between struts. The scaffold 600 may include two or more rigid rings 630 and an elastic ring or other flexible configuration (e.g., corrugations) 640 that allow for mechanical expansion of the scaffold 630 (e.g., with stretching of the surrounding or segmented membrane). An anchoring structure 650, analogous to the anchor 520 discussed above, permits fluid communication between the interior of the scaffold 600. The chamber membrane seals at or around the anchor 650.

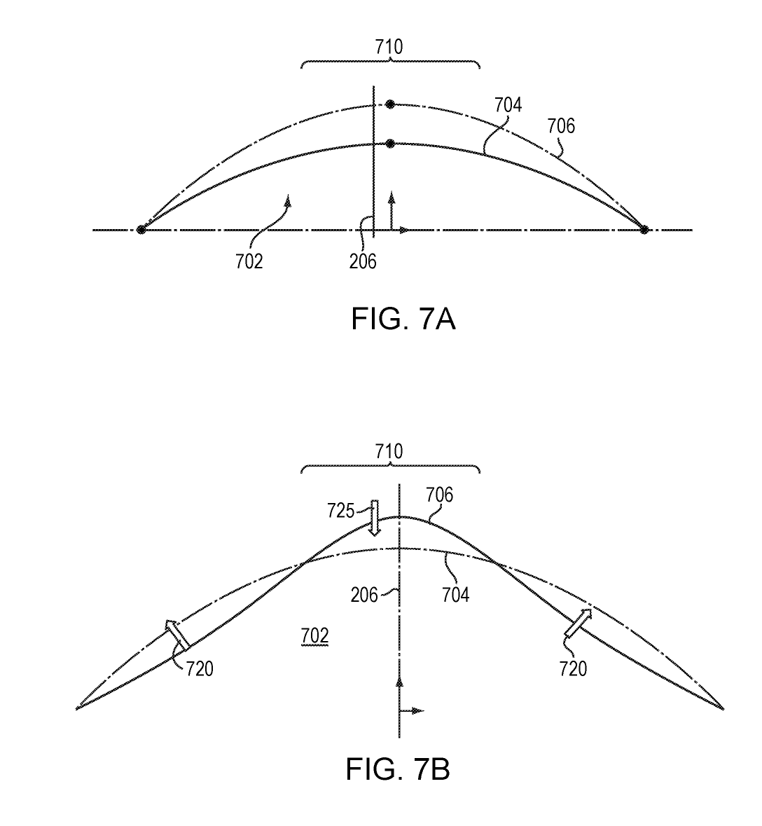

Alternatively or in addition to above-described structures, one or more of the flexible membranes may be designed to amplify or reverse the impact of additional fluid in the inner region 102. Conventionally, as shown in FIG. 7A, an increase in fluid pressure in the inner AIOL region 702 would expand or displace the surface of the membrane from the conformation indicated at 704 to that indicated at 706. This transition to a more convex shape of the optical zone 710 would provide more optical power.

As shown in FIG. 7B, it is also possible for an increase in fluid pressure in the inner region 702 to distort the membrane surface so that it provides less optical power. To promote this behavior, the membrane is formed (e.g., by molding, casting, etc.) to a rest shape having both convex and concave regions. An increase in fluid pressure in the interior 702 alters the membrane surfaces differently. Within the optical zone 710, the membrane expands vertically and remains convex but provides less optical power, whereas the membrane regions outside the optical zone 710 become concave; that is, the inner region decreases in convexity while the outer concentric region decreases in concavity. The degree or rate of concave curvature change, indicated at 720, is less than the degree or rate of convex curvature change, indicated at 725. As a result, the interior region 702 increases in volume. Similarly, other configurations with varying degrees of concavity in the optical zone 710 and surrounding an inner region 702 may be implemented to provide a desired range of accommodation and sensitivity. These may be defined by selecting the radial position of the inflection point (actually, a circular line or other closed shape where the membrane characteristics transition) and the relative thicknesses and elasticities of the concave and convex membrane regions.

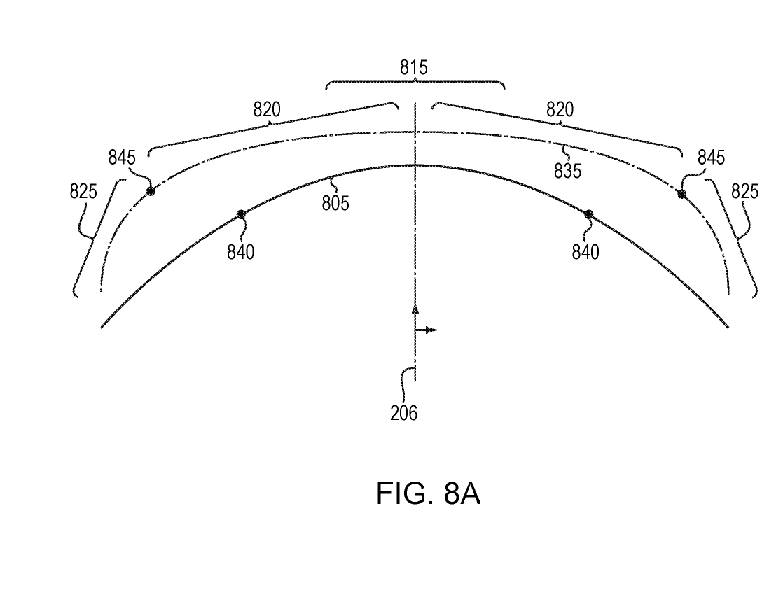

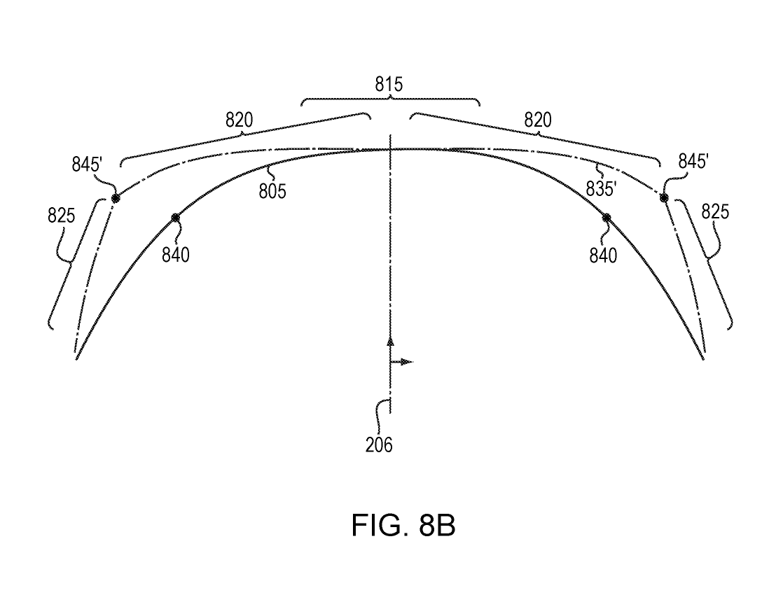

The membrane can be designed to exhibit multiple transitions to provide a desired overall contour. In FIGS. 8A and 8B, the depicted AIOL membrane 805 has a plurality of radially sequential regions 815 (corresponding to the center of the optical zone), 820, 825. Again, each of these regions may have different membrane thicknesses and/or elasticities. In the embodiment shown in FIG. 8A, as the membrane 805 expands into a conformation 835, the transition line 840 travels to the position indicated at 845, which lies above the line 840; thus, the membrane 805 expands along both vertical and horizontal dimensions. By contrast, in the embodiment depicted in FIG. 8B, expansion of the membrane 805 into the conformation 835′ involves travel of the transition line 840 in an almost entirely horizontal direction to the line 845′, so the concavity (and hence optical power) of the lens is largely unchanged.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An intraocular lens comprising:
a membrane defining a central chamber for containing an optical fluid and, when filled, to provide vision correction when implanted in a patient's eye, the central chamber having an optical axis extending through a vision-correcting optical zone of the central chamber and a peripheral region at least partially surrounding the optical zone;
a membrane defining at least one peripheral chamber at least partially surrounding the central chamber along the peripheral region thereof and outside the optical zone, the peripheral chamber being in fluid communication with the central chamber; and
associated with the at least one peripheral chamber, a shape-retention member for resisting collapse of the associated peripheral chamber in response to external force.

2. The intraocular lens of claim 1, wherein the shape-retention member is at least one spring.

3. The intraocular lens of claim 2, wherein the at least one spring extends from an anterior inner surface of the intraocular lens to a posterior inner surface of the intraocular lens.

4. The intraocular lens of claim 3, wherein the spring is oriented parallel to the optical axis of the lens.

5. The intraocular lens of claim 1, wherein the shape-retention member is at least one buckling member.

6. The intraocular lens of claim 1, wherein the shape-retention member is a hinged arrangement of struts.

7. The intraocular lens of claim 6, wherein the struts comprise living hinges.

8. The intraocular lens of claim 6, further comprising an anchor integral with the peripheral-chamber-defining membrane to which at least one of the struts is affixed.

9. The intraocular lens of claim 6, wherein the struts are configured to form a three-dimensional scaffold with the peripheral-chamber-defining membrane stretched thereover.

10. The intraocular lens of claim 1, wherein the shape-retention member retains the associated peripheral chamber in an ellipsoidal configuration.

11. The intraocular lens of claim 1, wherein the shape-retention member is metal.

12. The intraocular lens of claim 1, wherein the shape-retention member is polymeric.

13. The intraocular lens of claim 1, wherein the shape-retention member is within the associated peripheral chamber.

14. The intraocular lens of claim 1, wherein the shape-retention member surrounds the associated peripheral chamber.

15. The intraocular lens of claim 1, wherein the shape-retention member maintains the peripheral chamber at either of two conformal states.

16. An intraocular lens comprising:
a membrane defining a central chamber for containing an optical fluid and, when filled, to provide vision correction when implanted in a patient's eye, the central chamber having an optical axis extending through a vision-correcting optical zone of the central chamber and a peripheral region at least partially surrounding the optical zone;
a membrane defining at least one peripheral chamber at least partially surrounding the central chamber along the peripheral region thereof and outside the optical zone, the peripheral chamber being in fluid communication with the central chamber.
wherein the membrane defining the central chamber comprises a plurality of concentric regions having different expansion properties.

17. The intraocular lens of claim 16, wherein the membrane defining a central chamber becomes more convex and increases optical power in response to increasing pressure within the central chamber.

18. The intraocular lens of claim 16, wherein the membrane defining a central chamber becomes less convex and decreases optical power in response to increasing pressure within the central chamber.

19. The intraocular lens of claim 16, wherein, in response to increasing pressure within the central chamber, a first concentric region increases in convexity and a second concentric region increases in concavity.

20. The intraocular lens of claim 16, wherein, in response to increasing pressure within the central chamber, a first concentric region decreases in convexity and a second concentric region decreases in concavity.

21. The intraocular lens of claim 16, wherein the concentric regions have different thicknesses.

22. The intraocular lens of claim 16, wherein the concentric regions have different elasticities.
